(19) United States
(12) Patent Application Publication
(10) Pub. No.: US 2022/0323426 A1
(43) Pub. Date: Oct. 13, 2022

(54) CHOLINERGIC POTENTIATION OF BINOCULAR VISION

(71) Applicants: THE ROYAL INSTITUTION FOR THE ADVANCEMENT OF LEARNING / MCGILL UNIVERSITY, Montreal (CA); UNIVERSITE DE MONTREAL, Montreal (CA)

(72) Inventors: Robert F. HESS, Montreal (CA); Jacob SHEYNIN, Fair lawn, NJ (US); Elvire VAUCHER, Montréal (CA)

(21) Appl. No.: 17/634,285

(22) PCT Filed: Aug. 12, 2020
(86) PCT No.: PCT/CA2020/051104

§ 371 Date: Feb. 10, 2022

Related U.S. Application Data
(60) Provisional application No. 62/885,949, filed on Aug. 13, 2019.

(51) Int. Cl. A61K 31/445; A61P 27/02

(52) U.S. Cl. CPC A61K 31/445; A61P 27/02

(57) ABSTRACT

The present disclosure concerns the use of a cholinesterase inhibitor for improving binocular function and, in some embodiments, lessening an imbalance of binocular vision by promoting fusional ability and/or reducing interocular inhibition in a subject. The cholinesterase inhibitor canbe used to treat or alleviate the symptoms of amblyopia or diplopia. The cholinesterase inhibitor can be a specific and reversible acetylcholinesterase inhibitor, such as, for example, donepezil (also referred to as donepezil or Aricept®).

### CROSS-REFERENCE TO RELATED APPLICATIONS

This application claims priority from U.S. provisional patent application 62/885,949 filed on Aug. 13, 2019 and herewith incorporated in its entirety.

### TECHNOLOGICAL FIELD

The present disclosure concerns therapeutic agents capable of improving binocular vision and promoting binocular fusion.

### BACKGROUND

Binocularity, a defining feature of human vision that enables stereopsis, is predicated on the ability to combine inputs from the two eyes to create a singular representation of the visual world in depth. Binocular integration occurs in layer 2/3 in the primary visual cortex (V1), where inhibitory lateral connections control monocular inputs from the thalamorecipient layer 4 (Basgöze et al., 2018). Previous work has implicated the endogenous neurotransmitter acetylcholine (ACh) in the excitatory/inhibitory (E/I) balance of V1, modulating the gain of thalamocortical synapses in layer 4c while also inhibiting intracortical interactions (Disney et al., 2012; Obermayer et al., 2017).

It would be highly desirable to be provided with a means to improve binocular function in normal subjects as well as in those afflicted by a binocular disorder so as to remodel their visual circuitry. Such means could be used, for example, for reducing eye strain that can occur due to prolong close work or an oculomotor imbalance. Such means could also be used in combination with ocular surgery or digital therapies to improve treatment outcome.

### BRIEF SUMMARY

The present disclosure concerns a cholinesterase inhibitor for improving binocular function in a subject, the use of a cholinesterase inhibitor for improving binocular function in a subject as well as the use of a cholinesterase inhibitor for the manufacture of a medicament for improving binocular function in a subject. In an embodiment, the subject has an imbalance in binocular vision. In another embodiment, the subject is afflicted by a binocular disorder (such as, for example, post-surgical disruptions to binocular function, oculomotor imbalances, amblyopia or diplopia). In certain embodiments, the subject (such as, for example, a subject experiencing strabismus) was subjected to ocular surgery for eye re-alignment. In additional embodiments, the subject has experienced an eye strain due to a binocular imbalance. In some embodiments, the cholinesterase inhibitor is for use in combination with a training therapy to improve binocular function. In embodiments, the cholinesterase inhibitor is an acetylcholinesterase inhibitor. In some specific embodiments, the acetylcholinesterase inhibitor is donepezil or a pharmaceutically acceptable salt thereof. In some embodiments, donepezil is provided at a dosage of about 5 mg. In some embodiments, the cholinesterase inhibitor is for daily administration or intermittent administration. In additional embodiments, the cholinesterase inhibitor is for administration for at least one week, at least one month or for at least one year. In some embodiments, the subject has been determined being afflicted with an imbalance in binocular vision and/or a binocular disorder. In additional embodiments, the binocular function, the oculomotor imbalance, the fusional ability and/or the interocular inhibition of the subject has been measured prior to the use of the cholinesterase inhibitor. Alternatively or in combination, the binocular function, the oculomotor imbalance, the fusional ability and/or the interocular inhibition of the subject is intended to be measured after the use of the cholinesterase inhibitor.

In another aspect, the present disclosure provides a method for improving binocular function in a subject in need thereof. The method comprises administering an effective dose of a cholinesterase inhibitor to the subject so as to improve binocular function. In an embodiment, the subject has an imbalance in binocular vision and, in some embodiments, can be afflicted by a binocular disorder (such as an oculomotor imbalance, a post-surgical disruption to binocular function, amblyopia or diplopia). In an embodiment, the subject has been subjected to ocular surgery for eye re-alignment and/or the method further comprises performing ocular surgery for eye re-alignment in the subject. In an embodiment, wherein the subject has experienced an eye strain due to a binocular imbalance. In still a further embodiment, the subject has been submitted to a training therapy to improve binocular function and/or the method further comprises submitting the subject to a training therapy to improve binocular function. In some embodiments, the cholinesterase inhibitor is an acetylcholinesterase inhibitor. In yet additional embodiment, the acetylcholinesterase inhibitor is donepezil or a pharmaceutically acceptable salt thereof. In some specific embodiments, donepezil is administered at a dosage of about 5 mg. In embodiments, the method comprises daily or intermittently administering the cholinesterase inhibitor to the subject. In some further embodiments, the method comprises administering the cholinesterase inhibitor for at least one week, at least one month or at least one year to the subject. In an embodiment, the subject has been determined being afflicted with an imbalance in binocular vision and/or the method further comprises determining if the subject is being afflicted with an imbalance in binocular vision. In yet another embodiment, the subject has been determined being afflicted with a binocular disorder and/or the method further comprises determining if the subject is being afflicted with a binocular disorder. In still a further embodiment, the binocular function, the oculomotor imbalance, the fusional ability and/or the interocular inhibition of the subject has been measured prior to the administration of the cholinesterase inhibitor and/or the method further comprises measuring the binocular function, the oculomotor imbalance, the fusional ability and/or the interocular inhibition of the subject prior to the administration of the cholinesterase inhibitor. In yet another embodiment, the binocular function, the oculomotor imbalance, the fusional ability and/or the interocular inhibition of the subject is intended to be measured after the administration of the cholinesterase inhibitor and/or the method further comprises measuring the binocular function, the oculomotor imbalance, the fusional ability and/or the interocular inhibition of the subject after the administration of the cholinesterase inhibitor.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides an overview of the experimental design.

FIG. 1A. Each block consisted of two rivalry runs (where participants viewed left-tilted and right-tilted gratings presented individually to the two eyes) and two replay runs (where participants watched computer-generated videos of simulated binocular rivalry, presented identically to both eyes), each lasting 90 s.

FIG. 1B. Participants were instructed to continuously indicate via key-press whether they were seeing (I) the left eye's image, (r) the right eye's image, (I1r) a piecemeal mixture of the two images, or (m) a superimposed mixture of the two images.

FIG. 2 shows the effect of donepezil on binocular rivalry dynamics. Each row illustrates (1) scatter plots of the raw data observed by taking the mean of each dependent variable (A-E) across three binocular rivalry runs at baseline (x-axis) and after treatment (y-axis) for both the placebo and donepezil sessions; (2) a bar plot where each bar represents the average of three binocular rivalry blocks conducted 3 h after ingesting a donepezil/placebo pill, divided by the average of three identical rivalry blocks at baseline, averaged across participants; and (3) a scatter plot of donepezil's effect on each dependent variable obtained by subtracting the post/pre values of the placebo session from those of the donepezil session. Left column illustrates data for the median durations of the four percept types, while the right column illustrates data for the fraction durations of each percept type (A-D). Black asterisks between bars indicate significant differences observed with Tukey's HSD tests in the post/pre values between treatment conditions. Error bars are 95% confidence intervals (from 1000 bootstrapped samples) of the mean; **Bonferroni-corrected p<0.01, *p<0.05.

FIG. 2A shows the results obtained when using superimposition percepts.

FIG. 2B shows the results obtained when using piecemeal percepts.

FIG. 2C shows the results obtained when using aggregate mixed percepts.

FIG. 2D shows the results obtained when using exclusive percepts.

FIG. 2E shows the results obtained when using a rivalry rate.

FIG. 3 shows the effect of donepezil on replay rivalry criterion and response latency. To ensure any treatment effects were the result of changes in visual neural activity as opposed to changes in motor or executive function, a rivalry replay control experiment was implemented where participants watched computer-generated videos of simulated binocular rivalry, presented identically to both eyes. There were no observable differences between donepezil and placebo sessions in the response latency or mixed criterion, suggesting that changes in non-simulated binocular rivalry dynamics are attributed to ACh-induced changes in the network dynamics of visual cortex. See FIG. 2 for additional details regarding the plots.

FIG. 3A shows the measurement of the criterion for categorizing a physical stimulus as “mixed”.

FIG. 3B shows the measurement of the response latency for discriminating changes in the physical stimulus.

### DETAILED DESCRIPTION

The present disclosure concerns the use of a cholinesterase inhibitor for improving binocular function, binocular vision or binocular stability in subjects. Without wishing to be bound to theory, it is understood that the cholinesterase inhibitor is able to reduce interocular inhibition and/or promote binocular fusion (e.g., fusional ability) which in turn will lead to improved binocular vision and or binocular stability (for example, reducing or eliminating intermittent diplopia) in the subject. The cholinesterase inhibitor can be used in subjects having normal binocular function or having an imbalance of binocular vision (and being afflicted, in some embodiments, by a range of visual disorders, including, but not limited to an oculomotor imbalance, amblyopia or diplopia). As used herein, the expression “binocular function” or “binocular vision” or “binocular stability” refers to a subject's ability to combine images provided from both eyes to obtain a stable, single fused percept which has the benefits of an enlarged visual field, and 3D perception, postural stability (e.g., depth perception). When a subject is not able to optimally or satisfactorily combine images or is not able to combine images at all, or be able to combine images in a stable fashion, such subject experiences, visual confusion, binocular visual instability, fatigue (eye strain), simply monocular vision or diplopia, these are referred collectively to as imbalances in binocular vision. As used herein, the expression “oculomotor imbalance” (which may also be referred to as a phoria or heterophoria) is an imbalanced the oculomotor system in keeping the two eyes aligned horizontally and vertically when fusion of the images seen by the two eyes view is not present. People with higher oculomotor imbalances or phorias, can, after prolonged close work, experience discomfort, eyestrain or intermittent diplopia when those large phorias become uncompensated. Often prismatic correction is prescribed or incorporated with their present prescription. A stronger fusional system due to cholinergic enhancement could help these oculomotor imbalances remaining compensated and therefore not causing problems.

Cholinesterase inhibitors have previously been shown to enhance the brain's plasticity (e.g., the brain's ability to learn, see U.S. Pat. No. 4,895,841). However, it was later shown that donepezil, a specific acetylcholinesterase inhibitor, is not useful to improve monocular visual function as demonstrated using perceptual learning (Chung et al., 2017) and can even impede perceptual eye dominance changes that occur from the short term patching of one eye (Sheynin et al., 2019). In the context of some embodiments of the present disclosure, cholinesterase inhibitors are capable of lessening the imbalance of binocular vision by reducing interocular inhibition/improving excitatory combination. The expression “interocular inhibition” refers to a subject's ability not to consider only one of the two images provided by one of his two eyes to generate an image. By reducing interocular inhibition and or increasing excitatory combination, the cholinesterase inhibitor allows the subject to more optimally fuse both images into a single binocular percept. In the context of some embodiments of the present disclosure, cholinesterase inhibitors are capable of lessening the imbalance of binocular vision by promoting fusional ability. The expression “fusional ability” refers to a subject's ability to perceptually combine the two images provided each of his two eyes to generate a unitary binocular image. By promoting fusional ability, the cholinesterase inhibitor allows the subject to more optimally and more stably fuse both images into a single binocular percept.

The cholinesterase inhibitor can be used to improve binocular vision in a subject having normal binocular function. For example, the cholinesterase inhibitors can be used to simply improve the binocular vision and 3D sensitivity of subjects with normal vision for occupational needs, such as pilots, athletes, surgeons, etc. or to lessen an eye strain due to an imbalance in binocular function.

The cholinesterase inhibitor can be used to treat or alleviate the symptoms in a subject afflicted by a binocular disorder. As used herein, a “binocular disorder” refers to a pathological dysregulated binocular vision impairment in which the afflicted subject has suboptimal binocular combination of visual information. Symptoms of binocular disorders include, but are not limited to headaches, eye strain, eye pain, blurred vision, reduced stereopsis and double vision. Binocular disorders can be caused by a variety of conditions including but not limited to suppression of one eye's information, an uncompensated phoria, a tropia, any imbalance in the information from the two eyes due to cataracts, refractive surgery, visual pathology. In some embodiments, the cholinesterase inhibitors can be used to lessen an imbalance of binocular vision in subjects afflicted by amblyopia. In some embodiments, the cholinesterase inhibitors can be used to lessen an imbalance of binocular vision in subjects afflicted by diplopia (including, but not limited to intermittent diplopia). In other embodiments, the cholinesterase inhibitors can be used to lessen an imbalance of binocular vision in subjects experiencing an eye strain due to an uncompensated phoria. In additional embodiments, the cholinesterase inhibitors can be used to lessen an imbalance of binocular vision in subjects undergoing rehabilitation after an eye surgery.

The presence of an imbalance in binocular function and even of a binocular disorder in a subject can be determined by methods known in the art designed to measure the oculomotor imbalance, the degree of suppression between the eyes, the strength of fusion and the stereoscopic sensitivity.

### Cholinesterase Inhibitors

Cholinesterase inhibitors are a class of therapeutic agents capable of limiting or halting the metabolic breakdown of choline-containing molecules, including the neurotransmitter such as acetylcholine. By limiting the metabolic breakdown of acetylcholine, increased levels of this neurotransmitter are intended to accumulate in the synaptic cleft and mediate its effects on neuronal signal transmission, thus affecting binocular vision.

In humans, there are two types of cholinesterases: acetylcholinesterase and butyrylcholinesterase. The cholinesterase inhibitor can be selective or non-selective. A selective acetylcholinesterase inhibitor is capable of inhibiting acetylcholinesterase but not butyrylcholinesterase. A selective butyrylcholinesterase inhibitor is capable of inhibiting butyrylcholinesterase but not acetylcholinesterase. A non-selective cholinesterase inhibitor can inhibit both acetylcholinesterase and butyrylcholinesterase. The cholinesterase inhibitor can be a reversible or non-reversible inhibitor. In the context of the present disclosure, only reversible cholinesterase inhibitor are being used to potentiate binocular vision. In some embodiments, a single type of cholinesterase inhibitor is used for lessening interocular inhibition. In other embodiments, a combination of more than one type of cholinesterase inhibitors is used for lessening interocular inhibition. In some embodiments, a single type of cholinesterase inhibitor is used for promoting fusional ability. In other embodiments, a combination of more than one type of cholinesterase inhibitors is used for promoting fusional ability. In one embodiment, the cholinesterase inhibitor is a selective and reversible acetylcholinesterase inhibitor that is well tolerated by the subject. Selective reversible inhibitors include, but are not limited to, piperidine derivatives, e.g. donepezil, alkaloids, e.g. galantamine, and benzylammoniums, e.g. ambenonium.

In a specific embodiment, the cholinesterase inhibitor is a non-selective reversible inhibitor. Non-selective reversible inhibitors include, but are not limited to, carbamate-containing compounds, e.g. rivastigmine, physostigmine, pyridostigmine, pyridine derivatives, e.g. tacrine, 7-methoxytacrine, aminopyridazines, e.g. minaprine, phenylammoniums, e.g. edrophonium, phenol ethers, e.g. gallamine triethiodide, and alkaloids, e.g. huperzine A.

In an alternative embodiment, the cholinesterase inhibitor is a non-selective irreversible inhibitor. Irreversible inhibitors include, but are not limited to, organophosphorus compounds, e.g. malathion, echothiophate, trichlorfon, and isoflurophate. Irreversible inhibition by organophosphorus compounds can be reversed by treatment with pyridinium derivatives, such as, pralidoxime.

In a specific embodiment, the acetylcholinesterase inhibitor can be a plant extract or derived from a plant extract. In some embodiments, the extract is an alkoidal, aqueous, buthanolic, chloroform, dichloromethane, ethanolic, ethyl acetate, hexanic, hydroalcoholic, or methanolic fraction or extract of the plant or a plant part. The extract can be obtained from the entire plant and/or a plant part such as a bulb, a root, a stem, a flower or another aerial part. Families and plant species with anti-cholinesterase activity include, but are not limited to, Scadoxus puniceus (L.) Friis & Nordal (Amaryllidaceae), Lannea schweinfurthii Engl. (Anacardiaceae), Carpolobia lutea G. Don (Polygalaceae), Xysmalobium undulatum (L.), W. T. Aiton (Apocynaceae), Phlegmariurus tetragonus (Hook. & Grey.), B. ∅llg (Lycopodiaceae), Esenbeckia leiocarpa Engl. (Rutaceae), Melissa officinalis L. (Lamiaceae), Crinum bulbispermum (Burm. f.), Milne-Redh. & Schweick (Amaryllidaceae), Morus alba L. (Moraceae), Angelica decursiva (Miq.), Franch. & Say. (Apiaceae), Buchanania axillaris (Desr.), Ramamoorthy (Anacardiaceae), Salvia miltiorrhiza Bunge (Lamiaceae), Huperzia serrata (Thunb.), Trevis (Lycopodiaceae), Berberis aetnensis C. Presl (Berberidaceae), Senna obtusifolia (L.), H. S. Irwin & Barneby (Leguminosae), Zanthoxylum davyi Waterm (Rutaceae), Ziziphus mucronata Willd. (Rhamnaceae), Crinum bulbispermum (Burm. f.), Berberis libanotica Ehrenb. ex C. K., Schneid, (Berberidaceae), Zephyranthes carinata (Amaryllidaceae), Crinum jagus (J. Thomps.), Dandy (Amaryllidaceae), Adenia gummifera (Harv.), Harms (Passifloraceae), Ochna obtusata DC. (Ochnaceae), Gossypium herbaceum L. (Malvaceae), Hippeastrum puniceum (Lam.), Voss (Amaryllidaceae), Hemidesmus indicus (L.), R. Br. Ex, Schutt (Apocynaceae), Ficus sur Forssk (Moraceae), Rumex hastatus D. Don (Polygonaceae), Acalypha alnifolia Klein ex Willd (Euphorbiaceae), Olax nana Wall. ex Benth (Olacaceae), Persicaria hydropiper (L.), Delarbre (Polygonaceae), Crinum bulbispermum (Burm. f.), Huperzia brevifolia (Grey. & Hook.), Holub (Lycopodiaceae), Piper capense L. f. (Piperaceae), Searsia mysorensis (G. Don), Moffett (Anacardiaceae), Huperzia squarrosa (G. Forst.), Trevis (Lycopodiaceae), Scabiosa arenaria Forssk (Caprifoliaceae), Pavetta indica L. (Rubiaceae), Polygonum hydropiper L. (Polygonaceae), Huperzia compacta (Hook.), Trevis (Lycopodiaceae), Jatropha gossypifolia L. (Euphorbiaceae), Stemona sessilifolia (Miq.), Miq. (Stemonaceae), Buchanania axillaris (Desr.), Elatostema papillosum Wedd (Urticaceae), and Nelumbo nucifera Gaertn. (Nelumbonaceae).

In a specific embodiment, the cholinesterase inhibitors is an acetylcholinesterase inhibitor. The acetylcholinesterase inhibitor can be a specific inhibitor, e.g., only capable of significantly inhibiting acetylcholinesterase. On the other hand, the acetylcholinesterase inhibitor can be a non-specific inhibitor, e.g., capable of significantly inhibiting acetylcholinesterase and at least one other cholinesterase. The acetylcholinesterase inhibitor can be a reversible inhibitor (e.g., is not covalently bound to the enzyme). In a specific embodiment, the cholinesterase inhibitor is a specific and reversible acetylcholinesterase inhibitor, such as, for example, donepezil (also referred to as donepezil or Aricept®), galantamine (also referred to as galantamine extended release (ER), galantamine hydrobromide, auro-galantamine ER and Apo-galantamine), 1,3-bis[5(diethyl-o-nitrobenzylammonium)pentyl]-6-methyluracil dibromide (also known as C547), ambenonium (also known as ambenonum, ambenonium base, and mytelase) or rivastigmine.

In other embodiments, the acetylcholinesterase inhibitors can be combined used alone or in combination with another therapeutic agent (which is not a cholinesterase inhibitor). In some embodiments, the cholinesterase inhibitor can be used in combination with a N-methyl-D-aspartate receptor antagonist to improve cognition. In additional embodiments, acetylcholinesterase inhibitors are used in conjunction with immune modulators to treat skeletal muscle weakness seen in myasthenia gravis.

In some embodiments, the cholinesterase inhibitor, including the acetylcholinesterase inhibitor, can be provided in the form of a pharmaceutically acceptable salt. The expression “pharmaceutically acceptable salts” refers to conventional acid-addition salts or base-addition salts that retain the biological effectiveness and properties of the cholinesterase inhibitor described herein. The pharmaceutically acceptable salts are formed from suitable non-toxic organic or inorganic acids or organic or inorganic bases. Sample acid-addition salts include those derived from inorganic acids such as hydrochloric acid, hydrobromic acid, hydroiodic acid, citric acid, sulfuric acid, sulfamic acid, phosphoric acid, and nitric acid, and those derived from organic acids such as p-toluenesulfonic acid, tartaric acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as e.g., tetramethylammonium hydroxide. The chemical modification of a cholinesterase inhibitor into a salt is a well-known technique which is used in attempting to improve properties involving physical or chemical stability, e.g., hygroscopicity, flowability or solubility of compounds. In an embodiment, when the cholinesterase inhibitor is an acetylcholinesterase inhibitor, such as donepezil, the pharmaceutically acceptable salt can be an hydrochloric salt. In another embodiment, when the cholinesterase inhibitor is a dual butyrylcholinesterase inhibitor and an acetylcholinesterase inhibitor, such as rivastigmine, the pharmaceutically acceptable salt can be a tartrate salt.

The cholinesterase inhibitor can be provided as a pharmaceutical composition, e.g., in the form of a (therapeutically) effective amounts (dose) of the cholinesterase inhibitor together with pharmaceutically acceptable diluents, preservatives, solubilizers, emulsifiers, adjuvants and/or carriers. The expression “effective amount” refers to an amount (dose) effective in mediating an effect to a subject, e.g., lessening of an imbalance of binocular vision by reducing interocular inhibition and/or promoting fusional ability or binocular stability in the subject. The expression “therapeutically effective amount” refers to an amount (dose) effective in mediating a therapeutic benefit to a subject (for example treatment and/or alleviation of symptoms of a binocular disorder). It is also to be understood herein that a “therapeutically effective amount” may be interpreted as an amount giving a desired therapeutic effect, either taken in one dose or in any dosage or route, taken alone or in combination with other therapeutic agents.

When the cholinesterase inhibitor is donepezil, the therapeutically effective amount, provided in a single dose tablet, can be between about 1 and 25 mg. In an embodiment, the therapeutically effective amount can be at least about 1, 2, 3, 4, 5, 6, 7, 8, 9, 10, 11, 12, 13, 14, 15, 16, 17, 18, 19, 20, 21, 22, 23 or 24 mg. In another embodiment, the therapeutically effective amount can be no more than 25, 24, 23, 22, 21, 20, 19, 18, 17, 16, 15, 14, 13, 12, 11, 10, 9, 8, 7, 6, 5, 4, 3 or 2 mg. In still another embodiment, the therapeutically effective amount can be between about 1, 2, 3, 4, 5, 6, 7, 8, 9, 10, 11, 12, 13, 14, 15, 16, 17, 18, 19, 20, 21, 22, 23 or 24 mg and about 25, 24, 23, 22, 21, 20, 19, 18, 17, 16, 15, 14, 13, 12, 11, 10, 9, 8, 7, 6, 5, 4, 3 or 2 mg. In yet another embodiment, the therapeutically effective amount can be about 5 mg. In yet another embodiment, the therapeutically effective amount can be about 10 mg.

When the cholinesterase inhibitor is rivastigmine, the therapeutically effective amount, provided in a single dose capsule, can be between 0.5 and 20 mg. In an embodiment, the therapeutically effective amount can be at least about 0.5, 1, 2, 3, 4, 5, 6, 7, 8, 9, 10, 11, 12, 13, 14, 15, 16, 17, 18 or 19 mg. In another embodiment, the therapeutically effective amount can be no more than 20, 19, 18, 17, 16, 15, 14, 13, 12, 11, 10, 9, 8, 7, 6, 5, 4, 3, 2 or 1 mg. In still another embodiment, the therapeutically effective amount can be between about 0.5, 1, 2, 3, 4, 5, 6, 7, 8, 9, 10, 11, 12, 13, 14, 15, 16, 17, 18 or 19 mg and about 20, 19, 18, 17, 16, 15, 14, 13, 12, 11, 10, 9, 8, 7, 6, 5, 4, 3, 2 or 1 mg. In yet another embodiment, the therapeutically effective amount can be about 1.5 mg. In still another embodiment, the therapeutically effective amount can be about 3 mg. In still another embodiment, the therapeutically effective amount can be about 4.5 mg. In still another embodiment, the therapeutically effective amount can be about 6 mg.

When the cholinesterase inhibitor is rivastigmine, the therapeutically effective strength of an oral solution, can be between 1 and 10 mg/ml. In an embodiment, the therapeutically effective strength can be at least about 1, 2, 3, 4, 5, 6, 7, 8 or 9 mg/ml. In another embodiment, the therapeutically effective strength can be no more than 10, 9, 8, 7, 6, 5, 4, 3 or 2 mg. In still another embodiment, the therapeutically effective strength can be between about 1, 2, 3, 4, 5, 6, 7, 8 or 9 mg/ml and about 10, 9, 8, 7, 6, 5, 4, 3 or 2 mg/ml. In yet another embodiment, the therapeutically effective strength can be about 2 mg/ml.

As indicated above, pharmaceutical compositions comprising the cholinesterase inhibitor can include a pharmaceutically acceptable carrier. A pharmaceutically acceptable carrier refers to an acceptable carrier that may be administered to a subject, together with a cholinesterase inhibitors of this disclosure, and which does not reduce or abolish the pharmacological activity thereof. A pharmaceutical carrier is generally selected to provide for the desired bulk, consistency, etc., when combined with components of a given pharmaceutical composition, in view of the intended administration mode. When the pharmaceutical composition is a solid formulation, typical pharmaceutical carriers include, but are not limited to binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycotate, etc.); and wetting agents (e.g., sodium lauryl sulphate, etc.). When the pharmaceutical composition is a solution or a suspension, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

The cholinesterase inhibitor is administered at a dose/strength and for a specific amount of time (regimen) necessary to lessen the imbalance of binocular vision by reducing the interocular inhibition and/or promoting fusional ability and binocular stability in the subject. In some embodiments, the cholinesterase inhibitor is administered daily or in a formulation that would provide a daily dose (e.g., long term release for example). In some embodiments, the cholinesterase inhibitor can be administered daily for or over a period of at least 1, 2, 3, 4, 5, 6, 7, 8, 9, 10 weeks or more In some embodiments, the cholinesterase inhibitor can be administered daily for or over a period of at least 1, 2, 3, 4, 5, 6, 7, 8, 9, 10 months or more. In some embodiments, the cholinesterase inhibitor can be administered daily for or over a period of at least 1, 2, 3, 4, 5, 6, 7, 8, 9, 10 years or more. In some additional embodiments, the cholinesterase inhibitor can be administered intermittently, e.g., daily for or over a specific first period of time followed by a second period in which the cholinesterase inhibitor is not administered to the subject followed by a third period of time in which the cholinesterase inhibitor is administered daily. The first period of time can last for at least 1, 2, 3, 4, 5, 6, 7, 8, 9, 10 weeks or more. The first period of time can last for at least 1, 2, 3, 4, 5, 6, 7, 8, 9, 10 months or more. The first period of time can last for at least 1, 2, 3, 4, 5, 6, 7, 8, 9, 10 years or more. The second period of time can last for at least 1, 2, 3, 4, 5, 6, 7, 8, 9, 10 weeks or more. The second period of time can last for at least 1, 2, 3, 4, 5, 6, 7, 8, 9, 10 months or more. The second period of time can last for at least 1, 2, 3, 4, 5, 6, 7, 8, 9, 10 years or more. The third period of time can last for at least 1, 2, 3, 4, 5, 6, 7, 8, 9, 10 weeks or more. The third period of time can last for at least 1, 2, 3, 4, 5, 6, 7, 8, 9, 10 months or more. The third period of time can last for at least 1, 2, 3, 4, 5, 6, 7, 8, 9, 10 years or more.

The cholinesterase inhibitors can be administered by any know route in any formulation that would allow the inhibitors to reach the brain. In an embodiment, the cholinesterase inhibitor can be administered nasally (via the nasal route). In a specific embodiment, the cholinesterase inhibitor can be administered orally. In an embodiment, the composition is adapted for delivery orally or sublingually (via the oral route), intravenously (via an intravenous route), parenterally (via a parenteral route), subcutaneously or transdermally (via cutanous route), intramuscularly (via a muscular route), intracranial (via a cranial route), intraorbital (via an orbital route), ophthalmic (via an ocular route), intraventricular (via a ventricular route), intracapsular (via a capsular route), intraspinal (via a spinal route), intrathecally, intracisternally, intraperitoneally, and intranasally (via a nasal route). In some embodiments, the cholinesterase inhibitor is formulated for oral administration.

### Therapeutic Uses and Methods

The cholinesterase inhibitors of the present disclosure can be used or administered in order to achieve an improvement in binocular function or a reduction in an imbalance of binocular function (in some embodiments, by reducing interocular inhibition and/or promoting fusional ability and or binocular stability in a subject). The subject can be a mammal, for example a human. The subject can be a pediatric subject, an infant, a pediatric subject, a teenager subject or an adult subject. The cholinesterase inhibitors can be used to treat or alleviate the symptoms of a binocular disorder. These expressions “treatment” and “alleviation of symptoms” refer to the ability of the cholinesterase inhibitor to limit the development, progression and/or symptomology of a binocular disorder. Binocular disorders come in many forms ranging from the a mild imbalance that might lead to headaches and eye strain (asthenopia) to a complete loss of binocular vision in such cases as amblyopia (e.g., suppression of the vision of one eye) or diplopia (e.g., double vision). Another form of binocular dysfunction is where the brain processes each eye's image but is unable to combine these two images into a single binocular percept, this is called diplopia and can occur as an unfortunate side-effect of eye training or eye re-alignment surgery or spontaneously. It may be intermittent or in some rarer cases, constant. In some embodiments, the cholinesterase inhibitor is used for improving binocular vision in the treatment or the alleviation of symptoms of amblyopia in a subject. In some embodiments, the cholinesterase inhibitor is used for improving binocular vision in the treatment or the alleviation of symptoms of diplopia in a subject. In some additional embodiments, the cholinesterase inhibitor is used for improving or increasing binocular vision in the visual rehabilitation of a subject having been subjected to eye re-alignment surgery or refractive surgery. In some further embodiments, the cholinesterase inhibitor is used for improving binocular vision in a subject experiencing an eye strain due to a binocular imbalance. In some further embodiments the cholinesterase inhibitor is used in a subject with binocular vision to improve binocular functions such as 3D vision for occupational reasons.

The cholinesterase inhibitors of the present disclosure can be used in combination with a binocular vision therapy (also referred to as visual therapy). Binocular vision therapy can be conducted before, during and/or after the cholinesterase inhibitor is administered to the subject. In some embodiments, the binocular vision therapy is conducted once the cholinesterase inhibitor has been administered. Binocular vision therapy refers to a professionally-supervised (physicians, optometrists and the like), non-surgical and customized program of visual activities designed to correct certain vision problems and/or improve visual skills. Binocular vision therapy can include the use of lenses, prisms, filters and software-assisted visual activities. Other devices, such as balance boards, metronomes and non-computerized visual instruments also can play an important role in a customized vision therapy program. Overall, the goal of vision therapy is to treat vision problems that cannot be treated successfully with eyeglasses, contact lenses and/or surgery alone, and help people achieve clear, comfortable and stable binocular vision

The cholinesterase inhibitors of the present disclosure can be used in combination with a training therapy such as, for example a computer-assisted (digital) therapy. In an embodiment, the cholinesterase inhibitors can be used with a training therapy described in PCT Patent Application Serial Number PCT/CA2020/050051, as well as U.S. Ser. Nos. 8,057,036, 8,066,372 and 10,716,707 which are incorporated herewith in their entirety.

The cholinesterase inhibitors can be used in subjects having been previously determined to experience an imbalance in binocular function. Subjects experiencing imbalances in binocular function are more susceptible to benefit receiving cholinesterase inhibitors. As such, the therapeutic methods of the present disclosure can include a step of determining the presence or measuring imbalances in binocular function in the subject prior to the administration of the cholinesterase inhibitors and administering cholinesterase inhibitors to subjects experiencing such imbalances. In some embodiments, the therapeutic methods of the present disclosure can include a step of determining the presence or measuring imbalances in binocular function in the subject after to the administration of the cholinesterase inhibitors. This optional step could be used to determine if the administration of the cholinesterase inhibitor improve binocular function in the subjects and if additional therapy should be conducted.

The cholinesterase inhibitors can be used in subjects having been previously diagnosed with a binocular disorder. Subjects afflicted by a binocular disorder are more susceptible to benefit receiving cholinesterase inhibitors. As such, the therapeutic methods of the present disclosure can include a step of determining the presence of a binocular disorder prior to the administration of the cholinesterase inhibitors. In some embodiments, the therapeutic methods can also include a step of determining the presence and/or severity of the binocular disorder after the administration of the cholinesterase inhibitors. This optional step could be used to determine if the administration of the cholinesterase inhibitor improve binocular function in the subjects and if additional therapy should be conducted.

The cholinesterase inhibitors can be used in subjects having been previously determined to experience an imbalance in interocular inhibition. Subjects experiencing imbalanced interocular inhibition are more susceptible to benefit receiving cholinesterase inhibitors. As such, the therapeutic methods of the present disclosure can include a step of determining the presence or measuring interocular inhibition balance in the subject prior to the administration of the cholinesterase inhibitors and administering cholinesterase inhibitors to subjects experiencing interocular inhibition. In some embodiments, the therapeutic methods of the present disclosure can include a step of determining the presence or measuring interocular inhibition balance in the subject after to the administration of the cholinesterase inhibitors. This optional step could be used to determine if the administration of the cholinesterase inhibitor improve binocular function in the subjects and if additional therapy should be conducted.

The cholinesterase inhibitors can be used in subjects having been previously determined to experience an deficit in fusional ability leading to a deficit in binocular vision or an instability of binocular function. Subjects experiencing imbalanced in fusional ability are more susceptible to benefit receiving cholinesterase inhibitors. As such, the therapeutic methods of the present disclosure can include a step of determining the presence or measuring fusional ability/binocular balance in the subject prior to the administration of the cholinesterase inhibitors and administering cholinesterase inhibitors to subjects experiencing a decrease in fusional ability. In some embodiments, the therapeutic methods of the present disclosure can include a step of determining the presence or measuring fusional ability binocular balance in the subject after to the administration of the cholinesterase inhibitors. This optional step could be used to determine if the administration of the cholinesterase inhibitor improve binocular function in the subjects and if additional therapy should be conducted.

In some embodiments of the uses and methods of the present disclosure, it is also possible to determine the presence or measure interocular inhibition imbalance/fusional ability in subjects having received one or more doses of the cholinesterase inhibitors. This information can be used to determine if the cholinesterase inhibitors provide an effect on binocular vision in the subjects. In some embodiments, the step can be used to determine if a higher dose should be administered to the subject (because for example, interocular inhibition imbalance still occurs and/or fusional ability could be further promoted to improve binocular vision). In another embodiment, the step can be used to determine if the cholinesterase inhibitor should be administered for a longer period of time (because for example, interocular inhibition imbalance still occurs and/or fusional ability could be further promoted to improve binocular). In still a further embodiment, the step can be used to help the physician to decide to stop the use of cholinesterase inhibitors (because interocular inhibition imbalance is no longer present, fusional ability has been improved or no effects are observed in the subject). The present invention will be more readily understood by referring to the following examples which are given to illustrate the invention rather than to limit its scope.

### EXAMPLE

### Effect of Donepezil on Binocular Vision

In the present example, binocular rivalry, a sensitive probe of interocular dynamics (Tong et al., 2006) was used to characterize ACh's role in binocular integration. Importantly, mixed visibility during rivalry highlights periods when complete interocular suppression fails. On the contrary, exclusive visibility indicates instances of complete perceptual suppression, recently causally linked to enhanced GABAergic inhibition (Mentch et al., 2019). Consequently, the diverse phenomenology of binocular rivalry percepts constitutes an indirect assay of cortical E/I balance (Robertson et al., 2013, 2016; Van Loon et al., 2013; Mentch et al., 2019).

Using a double-blind placebo-controlled crossover design, it is herewith demonstrated that a single administration of the acetlycholinesterase inhibitor (AChEI) donepezil (5 mg, oral) strongly affects binocular rivalry dynamics, with important perceptual consequences. Cholinergic stimulation via donepezil enhances both the fraction and median duration of mixed visibility during rivalry, thereby reducing the amount of time one eye suppresses the other.

Simultaneously, ACh also reduced the rate of rivalry, another sensitive probe of cortical E/I balance (Robertson et al., 2013; Van Loon et al., 2013). The data presented in this example indicate that ACh plays a fundamental role in modulating binocular vision, providing new insights into the neurophysiological basis of human binocularity and on ACh's role in visual perception.

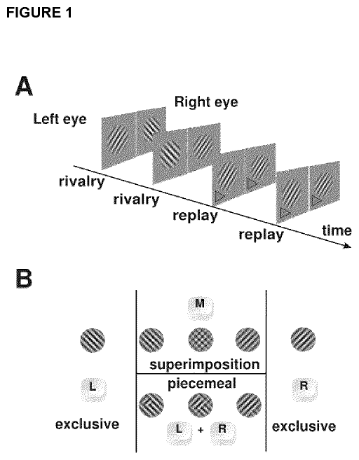

A total of 23 individuals completed two experimental sessions where binocular rivalry measurements were obtained before and after taking either donepezil or a placebo (lactose) pill. The binocular rivalry task consisted of a dichoptic stimulus where participants viewed a left-tilted grating in one eye and a right-tilted grating in the other for 90 s, continuously indicating via key-press whether they were seeing (1) the left eye's image, (2) the right eye's image, (3) a piecemeal mixture of the two images, or (4) a superimposed mixture of the two images (FIG. 1). This task was used to better characterize the mixed percepts while also encouraging participants not to miscategorize a mixed percept as exclusive.

Subjects. A total of 24 individuals enrolled in the study. One participant was excluded from the study due to a failure to complete the full experiment, therefore in sum, 23 individuals participated the study (13 males; age: 25±3; range: 20-32).

All subjects met the inclusion criteria (non-smoker, normal or corrected-to-normal visual acuity, normal stereo vision, no history of any neurologic or ocular diseases, no prescription medications). The body mass index range was specified as 17-26 kg/m² to ensure a similar distribution of the drug across subjects. All subjects were naive to the purpose of the experiment. A standard clinical and neurologic examination was performed. In addition, a stereoacuity test and an ECG recording were performed before the beginning of the experiment. Subjects were monitored for their safety during the experimental sessions with several blood pressure measurements taken.

Subjects gave written informed consent before the experiment. Data were collected and kept secure. Participants were enrolled, and their random allocation sequence was conducted by assigning drug/placebo in numbered containers. Subjects received financial compensation to cover travel expenses and time spent participating. The procedures were in accordance with the Helsinki Declaration of 2013 and the ethical standards of the Comité d'éthique de la recherche en santé, Université de Montréal, approval #12-084-CERES-P.

Apparatus. Each session took place in a quiet room with dim light. Visual stimuli were generated and controlled by an Apple MacBook Pro 2008 computer (MacOSX) running MATLAB R2012B (MathWorks, rrid:scr_001622) with the Psychophysics toolbox (Pelli, 1997; Brainard, 1997) rrid: scr_002881. Stimuli were presented on a y-corrected cathode ray tube monitor (LG) driven at a resolution of 1024×768 pixels, with a refresh rate of 75 Hz and a measured mean luminance of 60 cd/m⁻². Participants viewed stimuli through an eight-mirror modified Wheatstone stereoscope so that the left image was only seen by the left eye and the right image by the right eye. The position of the participant's head was stabilized with a chin rest at a viewing distance of 57 cm.

Donepezil pharmacological enhancement. Donepezil is a reversible, non-competitive, highly selective AChEI with a half-life of 80 h and a peak plasma level of 4.161.5 h after intake (Rogers et al., 1998); 5 mg of donepezil is the lowest prescribed dose which induces beneficial cognitive effects with very low adverse reaction incidence (Prvulovic and Schneider, 2014; Kang et al., 2014) and has produced several reported effects on adult vision (Silver et al., 2008; Rokem and Silver, 2010, 2013; Chamoun et al., 2017; Gratton et al., 2017). Importantly, although higher doses of donepezil may yield stronger effects on vision, a lower dose is more physiologically relevant to understanding the underlying, natural mechanisms of the visual system as it would not imbalance cortical neuromodulator levels as dramatically. Three hours before posttreatment testing, subjects ingested one cellulose capsule containing either 5-mg donepezil (auro-donepezil, Auro Pharma Inc) or lactose placebo, with water (Rokem and Silver, 2010; Chamoun et al., 2017; Sheynin et al., 2019a). The experimenter and subjects were naive to the experimental conditions.

Binocular rivalry task. A previously developed (Skerswetat et al., 2018) binocular rivalry task was adapted to quantify the fractions and median durations of exclusive, piecemeal, superimposition, and overall mixed percepts (for illustrations, see FIG. 1B). At the beginning of each session, participants were shown images on a document that illustrated the differences between the left-oriented, right-oriented, and superimposition versus piecemeal mixed percepts. Participants were told that they would see a dynamic stimulus during the experiment and that their task was to track what they were seeing, with particular attention to timeliness and accuracy.

Participants were given the option to continuously indicate whether they were seeing either (1) an exclusively left-tilted grating, (2) an exclusively right-tilted grating, (3) a superimposition mixed percept, or (4) a piecemeal mixed percept. Participants used three adjacent keys for the task, using the left to indicate exclusive left-tilt, right for right-tilt, a holding down a combination of the left and right keys for the piecemeal percepts, and the middle key for the superimposition percepts. It was specified that the criterion for exclusive percepts should be ˜90% left or right oriented.

Rivalry replay control. A rivalry replay control condition was used to characterize the criterion for categorizing a percept as mixed and to quantify the latency of binocular rivalry responses (Robertson et al., 2013, 2016). The replay control consisted of computer-generated videos presented binocularly, where the stimulus was oscillated from left-oriented gratings to right-oriented gratings along a continuous scale, such that the midpoint of this oscillation would produce a complete mixture of the two gratings. Each experimental block consisted of two binocular rivalry runs followed by two rivalry replay runs. Each replay run was generated using the time series extracted from the participants' data in a preceding binocular rivalry run within the same block, replaying the participant's rivalry dynamics so as to reduce the likelihood that the participant was aware of the fact that the replay control was a different experimental condition. The initial replay runs used to confirm a participant's key-mapping were generated artificially using a square function smoothened with a Gaussian kernel (window=30 s), where the exclusive percepts lasted exactly 2 s per phase and the intermediary mixed percept lasted 1 s per phase, and the percept oscillated evenly around a fully mixed percept. One of the replay runs in each block was generated with piecemeal mixed visibility as the mixed category, and the other with superimposition mixed visibility, so as to be able to characterize differences in criterion or response latency for these two different percept types. The average criterion used was extracted to categorize a percept as mixed by taking the mean value of the physical stimulus across all time points when the participant indicated they switched from exclusive to mixed visibility. The response latency was extracted by finding the time value corresponding to the minimum root mean square error (RMS) between the participant's responses and the physical stimulus. To obtain an estimate of the overall criterion and response latency for binocular rivalry (where piecemeal and superimposition both appear within a single run), the criterions and latencies were averaged across the two piecemeal and superimposition runs in each block.

Experimental design and statistical analysis. Participants (for details, see above, Subjects) were randomly allocated to either group 1 (donepezil first session, placebo second session) or group 2 (placebo first session, donepezil second session). Group assignment was counterbalanced across participants to control for possible session-order effects. The experimenter was not aware of the treatment condition of the two group assignments until after data collection was complete. For safety purposes, the experimenter recorded the participant's systolic blood pressure at baseline and monitored blood pressure levels throughout the experiment.

The general protocol of each experimental block is outlined in FIG. 1A. Each block consisted of two binocular rivalry runs followed by two rivalry replay runs, each lasting 90 s. It was confirmed that subjects correctly learned the key mapping corresponding to the percept categories by administering two replay runs at the beginning of every session, one run corresponding to the piecemeal mixed category, and the other to the superimposition category.

Each run (rivalry and replay) began with a dichoptic nonius cross presented inside a 3° oval surrounded by a black-and-white noise (1 cycle/degree) frame (side=10°). The observer was asked to make keypresses to adjust the position of the two frames to calibrate the optimal position for comfortable fusion. After confirmation, the participant was instructed to fixate at a fixation dot (0.2°) and place their hands on the appropriate keys to begin responding to the rivalry task. After a keypress, the dichoptic stimulus appeared and participants began responding to what they were observing on the monitor using the keypress instructions provided at the beginning of each session. Subsequent runs were initiated after a brief break where subjects viewed a mean-gray background screen. Subjects performed four experimental blocks before and after taking donepezil/placebo (after a 3 h drug incubation period). During the incubation period, subjects were instructed to keep both eyes open and do normal activities such as watching a movie or doing computer work in a well-lit room.

Baseline and posttreatment measurements were drawn from four experimental blocks. A mandatory 2-min break was implemented between each experimental block to prevent fatigue. The orientation of the gratings seen by the eyes during the rivalry runs was flipped between the two runs in each rivalry block to counterbalance possible orientation-eye biases and to interrupt any possible adaptation effects that would result in an increase in mixed visibility (Klink et al., 2010). The first experimental block was discarded in both baseline and posttreatment measurements to account for possible errors made in the beginning of the task.

Baseline and posttreatment measurements took place over the course of ˜30 min. The half-life of donepezil is 4.161.5 h after intake. Postdeprivation testing was begun at 3 h after drug administration to maximize the potency of the drug at the time of testing. During the drug incubation period, participants were instructed to keep their eyes open and do activities that require visual perception such as watching a movie, doing homework, or walking around the laboratory. Participants were also given a brief questionnaire before and after each experimental session that used a Likert scale (1-5) to quantify levels of arousal, along with two short answer questions to characterize whether they noticed any perceptual or psychosomatic differences between (1) the morning and afternoon sessions, and (2) between the two experimental sessions.

After completing the first session of an experiment, each participant was assigned a scheduled date to return for completing their second session. To ensure there was no residual effects from the previous session, all sessions were spaced at least one week apart from one another (Mean±SD=8±1.3 d).

Using the preprocessing methodology described in detail by Sheynin et al. (2019b), the key aspects of binocular rivalry dynamics corresponding to the overall fractions and median durations of (1) exclusive visibility, (2) piecemeal visibility, (3) superimposition visibility, and (4) aggregate (superimposition 1 piecemeal) mixed visibility were extracted. This preprocessing pipeline consisted of four stages: (1) remove the first and last percept states in the time series as well as all percept states shorter than 250 ms to obtain the preprocessed time series, (2) extract the distribution of percept phase durations for each state from the processed time series, (3) calculate the median and sum of these distributions to obtain the median and fraction duration of each of the states in each rivalry block. To obtain the fraction and median duration of aggregate mixed visibility, (4) adjacent piecemeal and superimposition percepts were concatenated in the original rivalry time-series data and re-executed the preprocessing paradigm mentioned above.

In addition, the overall rate of rivalry, defined as the total number of switches between the exclusive percepts divided by the run duration (Robertson et al., 2013, 2016) was extracted. In order to quantify the magnitude of the effect of the two treatment conditions relative to baseline, mean posttreatment values were divided by the mean baseline values to obtain post/pre ratios for each dependent variable across both experimental conditions. Furthermore, the effect of donepezil treatment was also evaluated by comparing post/pre values for variables obtained from the replay rivalry control condition. These were the following: (1) the criterion used to categorize a percept as mixed and (2) the response latency.

To ascertain the effect of donepezil on binocular rivalry dynamics, a repeated measures MANOVA of the drug effect on the post/pre values for the seven dependent variables obtained from the binocular rivalry experiment was conducted. Additionally, a separate repeated measures MANOVA of the drug effect on the two dependent variables obtained from the replay rivalry control condition was conducted to rule out the possibility that donepezil-induced effects in the data could be attributed to factors other than changes in binocular rivalry dynamics.

When the omnibus MANOVA test was significant, post hoc Bonferroni-corrected Tukey's

HSD tests was conducted on each dependent variable to determine which variables were affected by donepezil. In addition, a 95% confidence intervals (1000 bootstrapped samples, each drawing 23 subjects with replacement) was obtained for the mean drug effect for each dependent variable. All statistical analyses were conducted using IBM SPSS Statistics (version 24). This study was not preregistered. The datasets generated and analyzed during the completion of the present example are available online at https://github.com/ysheynin/Cholinergic_Modulation_Binocular_Vision.

A primary aim of the study was to evaluate the effect of donepezil on the diverse phenomenology of binocular rivalry percepts. To that end, the task allowed us to measure the median and fraction duration of piecemeal and superimposition mixed percepts during rivalry, as well as the median and fraction duration of aggregate mixed visibility and exclusive visibility. In addition, the rate of rivalry was also examined as another dependent variable.

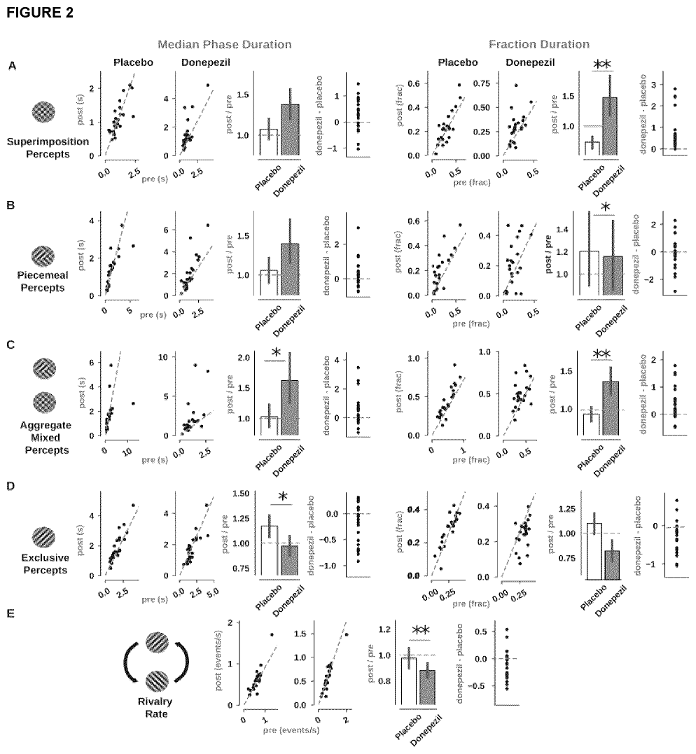

FIG. 2 illustrates the effect of donepezil on these aspects of binocular rivalry dynamics. A repeated measures MANOVA of the effect of session on the post/pre values for these variables was significant (F_{(1,22)}=2.68, p=0.04, η²ₚ=0.63, Wilks' λ=0.37), indicating that donepezil significantly altered binocular rivalry dynamics when compared with the placebo control.

An interesting pattern emerge was observed within the two mixed percept subcategories. Piecemeal percepts, where the two images appear combined as in a mosaic, are proposed to emerge from a reduction of the spatial coherence of interocular inhibition, whereas superimposition percepts, where the two component gratings appear overlaid as in a plaid, likely correspond to decreases in the gain of interocular inhibition (Klink et al., 2010; FIG. 1A). Cholinergic stimulation enhances the fraction visibility of superimposition percepts by 70% (p=0.001, 95% CI (Confidence Interval) [31%, 108%]; FIG. 2A, right), while simultaneously reducing the fraction of piecemeal percepts by 15% (p=0.03, 95% CI [9%, 30%]; FIG. 2B, right). Although not statistically significant, a different pattern emerge was observed for the median durations of these percepts. Both the median duration of piecemeal (M=49%, p=0.06, 95% CI [−2%, 81%]) and superimposition (M=54%, p=0.06, 95% CI [−3%, 110%]) percepts increased with administration of donepezil. Despite this inconsistency (which may be due to variance in the baseline data between the two sessions), these data suggest that

ACh predominantly modulates the overall gain of interocular inhibition and reduces the spatial coherence of the inhibition.

Furthermore, the results indicate that donepezil significantly enhanced the fraction of aggregate mixed visibility during rivalry by 43% (p=0.002, 95% CI [18%, 68%]; FIG. 2C, right) and likewise increased the median duration of mixed visibility by 20% (p=0.025, 95% CI [4%, 37%]; FIG. 2C, left). Increases in the fraction of mixed visibility were observed in 19 out of 23 participants, where eight of these individuals exhibited donepezil-induced increases of 0.50%. These changes were reciprocated in measures of exclusive visibility during rivalry, where donepezil reduces both the median duration of exclusive percepts by 78% (p=0.01, 95% CI [18%, 139%]; FIG. 2D, left) and the fraction of exclusive percepts by 59% (p=0.46, 95% CI [−220%, 103%]; FIG. 2D, right). The rate of binocular rivalry also decreased by 36% (p=0.002, 95% CI [13%, 54%]; FIG. 2E) in the donepezil condition relative to placebo, suggesting attenuation of cortical inhibition (Robertson et al., 2013; Van Loon et al., 2013; Mentch et al., 2019). Together, these results point to an ACh-induced increase in the visibility of mixed percepts during rivalry, likely due to a shift in favor of excitation.

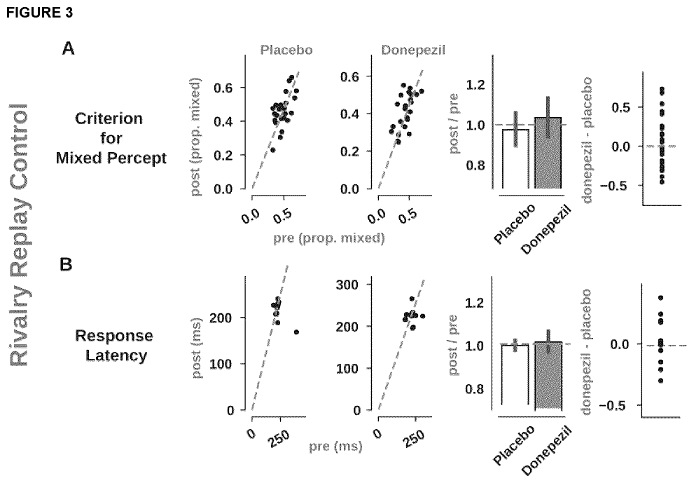

Despite the dramatic increase in the visibility of mixed percepts, these changes were not reflected in individuals' self-report of their experience after the experiment, nor during a control condition where rivalry playback videos were generated and the criterion used for categorizing a percept as mixed was measured. While mixed visibility increased substantially on donepezil, a MANOVA conducted on the criterion and response latency from the rivalry replay control experiment was not significant (F₍1,22)=1.4, p=0.04, η²ₚ=0.63, Wilks' λ=0.88), indicating that the perceptual changes observed in the donepezil condition cannot be caused by changes in subjects' response criteria (FIG. 3A) or response latency (FIG. 3B) and can only be attributed to changes in neural activity.

Together, these results point to an ACh-induced increase in the visibility of mixed percepts during rivalry, especially superimposed percepts, and likely due to a shift in favor of excitatory drive. Importantly, these findings cannot be attributed solely to an increase in the gain of purely monocular signals, as previous work has demonstrated that enhanced stimulus contrast, which increases monocular gain, actually reduces mixed visibility during rivalry (Hollins, 1980). The results are therefore aligned with the notion that cholinergic potentiation reduces the gain of interocular inhibition in layers 2/3 of V1.Likewise, the results align themselves with recent evidence published in (Mentch et al., 2019), which implicated GABAergic inhibition in gating perceptual awareness during binocular rivalry. While enhanced GABAergic transmission increases perceptual suppression and occasionally increases rivalry alternation rates, the data presented herein demonstrated, for the first time, that enhanced cholinergic activity reduces perceptual exclusivity during rivalry, decreases alternation rates, and thus has an opposite effect to GABA. In fact, the data demonstrate that superimposition, not piecemeal percepts, is maximally enhanced by donepezil. This is consistent with the idea that interocular inhibition is reduced and excitatory combination, strengthened. Moreover, the reduced rate of rivalry marked by increases in mixed visibility, as found herein, points to an excitation-dominant cortical response profile, opposed to an increased switch rate with increased GABAergic transmission. Combined, these findings lend additional support for previous models of binocular rivalry that implicated changes in E/I balance in binocular cortex to perceptual awareness during rivalry (Wilson, 2003; Brascamp et al., 2013; Said and Heeger, 2013).

Critically, the data presented herein implicate the cholinergic system in modulating the E/I balance of binocular visual cortex. Cholinergic fibers modulate various inhibitory circuits: feed-forward inhibition, lateral inhibition, and disinhibition (Obermayer et al., 2017), so the effect on GABAergic circuits induced by enhanced cholinergic transmission cannot be directly inferred. However, it is highly plausible, given the data presented herein, recent evidence from Mentch et al. (2019), as well as previous electrophysiological work from Krueger and Disney (2019), that the predominant cholinergic enhancement effect in V1 would reduce the inhibitory drive that enables ocular suppression, leading to increases in interocular interactions and the occurrence of mixed percepts.

### REFERENCES

Basgöze Z, Mackey A P, Cooper E A (2018) Plasticity and adaptation in adult binocular vision. Curr Biol 28:r1406-r1413.
Brainard D H (1997) The psychophysics toolbox. Spat Vis 10:433-436.
Brascamp J, Sohn H, Lee S H, Blake R (2013) A monocular contribution to stimulus rivalry. Proc Natl Acad Sci USA 110:8337-8344.
Chamoun M, Huppé-Gourgues F, Legault I, Rosa-Neto P, Dumbrava D, Faubert J, Vaucher E (2017) Cholinergic potentiation improves perceptual-cognitive training of healthy young adults in three dimensional multiple object tracking. Front Hum Neurosci 11:128-129.
Disney A A, Aoki C, Hawken M J (2012) Cholinergic suppression of visual responses in primate V1 is mediated by GABAergic inhibition. J Neurophysiol 108:1907-1923.
Gratton C, Yousef S, Aarts E, Wallace D L, D'Esposito M, Silver M A (2017) Cholinergic, but not dopaminergic or noradrenergic, enhancement sharpens visual spatial perception in humans. J Neurosci 37:4405-4415.
Hollins M (1980) The effect of contrast on the completeness of binocular rivalry suppression. Percept Psychophys 27:550-556.
Kang J I, Huppé-Gourgues F, Vaucher E (2014) Boosting visual cortex function and plasticity with acetylcholine to enhance visual perception. Front Syst Neurosci 8:172.
Klink P C, Brascamp J W, Blake R, Van Wezel R J A (2010) Experience-driven plasticity in binocular vision. Curr Biol 20:1464-1469.
Krueger J, Disney A A (2019) Structure and function of dual-source cholinergic modulation in early vision. J Comp Neurol 527:738-750.
Mentch J, Spiegel A, Ricciardi C, Robertson CE (2019) GABAergic inhibition gates perceptual awareness during binocular rivalry. J Neurosci 39:8398-8407.
Obermayer J, Verhoog M B, Luchicchi A, Mansvelder H D (2017) Cholinergic modulation of cortical microcircuits is layer-specific: evidence from rodent, monkey and human brain. Front Neural Circuits 11:100.
Pelli D G (1997) Pixel independence: measuring spatial interactions on a CRT display. Spat Vis 10:443-446.
Prvulovic D, Schneider B (2014) Pharmacokinetic and pharmacodynamic evaluation of donepezil for the treatment of Alzheimer's disease. Expert Opin Drug Metab Toxicol 10:1039-1050.
Robertson C E, Kravitz D J, Freyberg J, Baron-Cohen S, Baker C I (2013) Slower rate of binocular rivalry in autism. J Neurosci 33:16983-16991.
Robertson C E, Ratai E M, Kanwisher N (2016) Reduced GABAergic action in the autistic brain. Curr Biol 26:80-85.
Rogers C, Sukovaty P, Lee F, Rogers S L, Cooper N M, Sukovaty R, Pederson J E, Lee J N, Friedhoff L T, Rogers C, Sukovaty P, Lee F (1998) Pharmacokinetic and pharmacodynamic profile of donepezil HCl following multiple oral doses. Br J Clin Pharmacol 46:7-12.
Rokem A, Silver M A (2010) Cholinergic enhancement augments magnitude and specificity of visual perceptual learning in healthy humans. Curr Biol 20:1723-1728.
Rokem A, Silver M A (2013) The benefits of cholinergic enhancement during perceptual learning are long-lasting. Front Comput Neurosci 7:66.
Said C P, Heeger D J (2013) A model of binocular rivalry and cross-orientation suppression. PLoS Comput Biol 9:e1002991.
Sheynin Y, Chamoun M, Baldwin A S, Rosa-Neto P, Hess R F, Vaucher E (2019a) Cholinergic potentiation alters perceptual eye dominance plasticity induced by a few hours of monocular patching in adults. Front Neurosci 13:22-12.
Sheynin Y, Proulx S, Hess R F (2019b) Temporarymonocular occlusion facilitates binocular fusion during rivalry. J Vis 19:23-17.
Silver M A, Shenhav A, D'Esposito M, Esposito M D, D'Esposito M (2008) Cholinergic enhancement reduces spatial spread of visual responses in human early visual cortex. Neuron 60:904-914.
Skerswetat J, Formankiewicz M A, Waugh S J (2018) More superimposition for contrast-modulated than luminance-modulated stimuli during binocular rivalry. Vision Res 142:4051-914.
Tong F, Meng M, Blake R (2006) Neural bases of binocular rivalry. Trends Cogn Sci 10:502-511.
Van Loon A M, Knapen T, Scholte H S, St. John-Saaltink E, Donner T H, Lamme V A F (2013) GABA shapes the dynamics of bistable perception. Curr Biol 23:823-827.
Wilson H R (2003) Computational evidence for a rivalry hierarchy in vision. Proc Natl Acad Sci USA 100:14499-14503.
Chung S T L, Li R W, Silver M A, Levi D M. Donepezil Does Not Enhance Perceptual Learning in Adults with Amblyopia: A Pilot Study. Front Neurosci. 2017 Aug. 7;11:448.
U.S. Pat. No. 4,895,841

1-21. (Canceled)

22. A method for improving binocular function in a subject in need thereof, the method comprising administering an effective dose of a cholinesterase inhibitor to the subject so as to improve binocular function.

23. The method of claim 22, wherein the subject has an imbalance in binocular vision.

24. The method of claim 22, wherein the subject is afflicted by a binocular disorder.

25. The method of claim 24, wherein the binocular disorder is amblyopia, diplopia, a post-chirurgical disruption to binocular function or an oculomotor imbalance.

26. The method of claim 22, wherein the subject has been subjected to ocular surgery for eye re-alignment.

27. The method of claim 22, further comprising performing ocular surgery for eye re-alignment in the subject.

28. The method of claim 22, wherein the subject has experienced an eye strain due to a binocular imbalance.

29. The method of claim 22, wherein the subject has been submitted to a training therapy to improve binocular function.

30. The method of claim 22, further comprising submitting the subject to a training therapy to improve binocular function.

31. The method of claim 22, wherein the cholinesterase inhibitor is an acetylcholinesterase inhibitor.

32. The method of claim 31, wherein the acetylcholinesterase inhibitor is donepezil or a pharmaceutically acceptable salt thereof.

33. The method of claim 32, wherein donepezil is administered at a dosage of about 5 mg.

34. The method of claim 22 comprising daily administering the cholinesterase inhibitor to the subject.

35. The method of claim 22 comprising administering intermittently the cholinesterase inhibitor to the subject.

36. The method claim 22 comprising administering the cholinesterase inhibitor for at least one week to the subject.

37. The method claim 22 comprising administering the cholinesterase inhibitor for at least one month to the subject.

38. The method of claim 22 comprising administering the cholinesterase inhibitor for at least one year to the subject.

39. The method of claim 22, wherein the subject has been determined being afflicted with an imbalance in binocular vision.

40. The method of claim 22 further comprising determining if the subject is being afflicted with an imbalance in binocular vision.

41. The method of claim 22, wherein the subject has been determined being afflicted with a binocular disorder.

42. The method of claim 22 further comprising determining if the subject is being afflicted with a binocular disorder.

43. The method of claim 22, wherein the binocular function, the fusional ability, the oculomotor imbalance and/or the interocular inhibition of the subject has been measured prior to the administration of the cholinesterase inhibitor.

44. The method of claim 22 further comprising measuring the binocular function, the fusional ability, the oculomotor imbalance and/or the interocular inhibition of the subject prior to the administration of the cholinesterase inhibitor.

45. The method of claim 22, wherein the binocular function, the fusional ability, the oculomotor imbalance and/or the interocular inhibition of the subject is intended to be measured after the administration of the cholinesterase inhibitor.

46. The method of claim 22 further comprising measuring the binocular function, the fusional ability, the oculomotor imbalance and/or the interocular inhibition of the subject after the administration of the cholinesterase inhibitor.
